(19) United States
(12) Patent Application Publication
(10) Pub. No.: US 2026/0146227 A1
(43) Pub. Date: May 28, 2026

(54) Anisotropic Hydrogel and Preparation Method and Use thereof

(71) Applicant: ZHEJIANG UNIVERSITY, Hangzhou (CN)

(72) Inventors: Wenjun WANG, Hangzhou (CN); Hao WANG, Hangzhou (CN); Congyi XU, Hangzhou (CN); Haiqi CHEN, Hangzhou (CN); Tiange PAN, Hangzhou (CN); Lifen ZHANG, Hangzhou (CN); Enbo XU, Hangzhou (CN); Ruiling LV, Hangzhou (CN); Donghong LIU, Hangzhou (CN)

(73) Assignee: ZHEJIANG UNIVERSITY, Hangzhou (CN)

(21) Appl. No.: 19/403,204

(22) Filed: Nov. 27, 2025

(30) Foreign Application Priority Data
Nov. 28, 2024 (CN) 2024117251710

(51) Int. Cl. C12N 5/00; A23L 13/00; C12N 9/10

(52) U.S. Cl. CPC C12N 5/0062; A23L 13/00; C12N 9/1044; C12Y 203/02013; C12N 2513/00; C12N 2533/50; C12N 2533/54; C12N 2537/10

(57) ABSTRACT

Provided is an anisotropic hydrogel and its preparation method and use. In the preparation method of the anisotropic hydrogel, transglutaminase is added to introduce enzymatic chemical crosslinking, which, in combination with physical crosslinking, enables the hydrogel to be stretched to form anisotropy and stabilizes the resulting anisotropy. The surface of the anisotropic hydrogel exhibits an ordered and oriented structure. When applied to in vitro cell culture, it enables the cells to grow in a uniform direction. When the anisotropic hydrogel is used for preparing cultured meat, the resulting cultured meat presents a fibrous texture close to that of real meat. The preparation method of the anisotropic hydrogel is simple to operate. In the fibrous material dispersion, the fibrous material includes cellulose or protein isolate amyloid fibrils, and these raw materials are non-toxic and harmless, making them suitable for use in the food field.

### REFERENCE TO RELATED APPLICATIONS

This is an application claiming the benefit of priority to a Chinese Patent Application No. 202411725171.0, filed on Nov. 28, 2024, entitled “Anisotropic Hydrogel and Preparation Method and Use Thereof”, the disclosure of which is incorporated herein by reference in its entirety, including any appendices and attachments thereof, for all purposes.

### FIELD OF TECHNOLOGY

The present disclosure relates to the technical field of hydrogel preparation, and particularly to an anisotropic hydrogel and a preparation method and use thereof.

### BACKGROUND

Hydrogels, due to their good biocompatibility and high water content, have been widely applied in the fields of food science, tissue engineering, drug delivery, cell culture, etc. Hydrogels serve as functional materials in food processing, texture/structure regulation, and nutrient delivery. For example, hydrogels may be designed for encapsulating active components or regulating the texture and mouthfeel of food, thereby improving the stability and flavor of the food. In addition, hydrogels are also widely used for simulating food structures to study the physicochemical properties of food during processing and storage. In recent years, research related to hydrogel materials has focused on developing structures with anisotropic characteristics, which facilitates simulating the physical properties of food in different directions. Particularly in food texture/structure design, anisotropic hydrogels enable stimulation of mechanical responses of food in different chewing directions, thereby contributes to the consumer's chewing experience improvement. Such anisotropic hydrogel materials not only have significant application prospects in the development of novel foods but also provide new solutions for functional foods and personalized nutrition design.

Traditional methods for constructing anisotropic hydrogels include freezing, templating, electrospinning, etc. However, these methods often have the drawback of complex processing procedures.

### SUMMARY

The present disclosure provides an anisotropic hydrogel and a preparation method and use thereof. The preparation method of the present disclosure is simple to operate.

The present disclosure provides a preparation method of an anisotropic hydrogel, including:
1) providing a fibrous material dispersion;
2) mixing the fibrous material dispersion, a gelatin solution, and transglutaminase to obtain a precursor solution, and subjecting the precursor solution to pre-crosslinking and freeze-crosslinking in sequence, to obtain a crosslinked hydrogel;
3) mixing the crosslinked hydrogel with a first salt solution to perform salting-out and obtain a salting-out hydrogel;
4) stretching the salting-out hydrogel to obtain a stretched hydrogel; and
5) mixing the stretched hydrogel with a second salt solution and performing curing, to obtain the anisotropic hydrogel.

A physical crosslinking effect is present in the fibrous material dispersion.

In some embodiments, a fibrous material in the fibrous material dispersion includes cellulose or protein isolate amyloid fibrils.

When protein isolate amyloid fibrils are employed as the fibrous material in the fibrous material dispersion, a preparation method of an protein isolate amyloid fibril dispersion includes:
adding a protein isolate into an ethanol-water system, adjusting the pH to an acidic value, and performing water-bath heating, to obtain the protein isolate amyloid fibril dispersion.

The protein isolate is pea protein isolate or soy protein isolate.

A volume concentration of ethanol in the ethanol-water system is in a range of 0-50%.

The acidic pH value is in a range of 1.5-3.

The water-bath heating is carried out at 70-90° C. for 20-40 hours.

In some embodiments, when the protein isolate is soy protein isolate, after adjusting the pH to an acidic value and before performing water-bath heating, the preparation method of the protein isolate amyloid fibril dispersion further includes performing hydration and removing precipitates, wherein the hydration is carried out at 2-8° C. for 6-10 hours.

In some embodiments, in the precursor solution, a mass concentration of the fibrous material is in a range of 0.5-8%, a mass concentration of gelatin is in a range of 3-10%, and a mass ratio of transglutaminase to gelatin ranges from 1:1 to 1:12.

The pre-crosslinking is carried out at 40-60° C. for 2-30 minutes.

The freeze-crosslinking is carried out at 4° C. for 1-3 hours.

In some embodiments, a salt in the first salt solution includes one or more of sodium chloride, sodium sulfate, and sodium citrate. A mass concentration of the first salt solution is in a range of 15-40%.

The salting-out is carried out for 0.5-2 hours.

In some embodiments, the salting-out hydrogel is stretched to 1-6 times its original length.

In some embodiments, a salt in the second salt solution includes sodium citrate, and a mass concentration of the second salt solution is in a range of 15-40%.

The curing is carried out for 10-20 hours.

The present disclosure further provides an anisotropic hydrogel prepared by the preparation method described above.

The present disclosure further provides a use of the anisotropic hydrogel described above in the food field.

In some embodiments, the food field is cultured meat preparation.

The method of the present disclosure uses a fibrous material dispersion, in which the fibrous material imparts anisotropy to the hydrogel during subsequent stretching. Adding transglutaminase introduces enzymatic chemical crosslinking. The enzymatic chemical crosslinking, combined with physical crosslinking from salting-out, enables the hydrogel to be stretched to form anisotropy and stabilizes the resulting anisotropy, thereby enhancing the structural stability and mechanical performance of the hydrogel. The surface of the anisotropic hydrogel prepared by the method of the present disclosure exhibits an ordered and oriented structure. When applied to in vitro cell culture, the anisotropic hydrogel enables cells to grow in a uniform direction. Specifically, when used for the preparation of cultured meat, the resulting cultured meat presents a fibrous texture close to that of real meat. Moreover, the preparation method of the present disclosure is simple to operate.

Furthermore, the fibrous materials in the fibrous material dispersion used in the present disclosure, including cellulose or protein isolate amyloid fibrils, are non-toxic and harmless, making them suitable for the food field.

Additionally, when preparing the protein isolate amyloid fibril dispersion, the volume concentration of ethanol in the ethanol-water system can be used to control the morphology of the protein isolate amyloid fibrils, thereby enabling regulation of the microscopic morphology of the final hydrogel.

### BRIEF DESCRIPTION OF THE DRAWINGS

FIG. 1 is a scanning transmission electron microscopy image of the protein isolate amyloid fibrils in the dispersions obtained in Example 1.

FIG. 2 is a polarizing microscopy image of the anisotropic gelatin-protein isolate amyloid fibril hydrogel obtained in Example 2.

FIG. 3 is an optical photograph of the anisotropic gelatin-protein isolate amyloid fibril hydrogel obtained in Example 2 after incubating at 27° C. and 37° C. for different durations.

FIG. 4 is a stress-strain curve of the soy protein isolate amyloid fibril-salting-out hydrogel obtained in Example 3.

FIG. 5 is a scanning electron microscopy image of the anisotropic gelatin-pea protein isolate amyloid fibril hydrogel obtained in Example 4.

FIG. 6 is an optical photograph of the anisotropic gelatin-protein isolate amyloid fibril hydrogel obtained in Example 4 after incubating at 27° C. and 37° C. for different durations.

FIG. 7 is a cell morphology image obtained after cell culture on unstretched and stretched hydrogels in Example 4.

### DETAILED DESCRIPTION

### Definitions of Terms

Anisotropy refers to the phenomenon that a material exhibits different physical or chemical properties along different directions, where the physical or chemical properties include, but are not limited to, mechanical properties, swelling behavior, electrical conductivity, or thermal conductivity. Anisotropy is contrasted with isotropy, isotropy means that a material has identical properties in all directions.

The present disclosure provides a preparation method of an anisotropic hydrogel, including:
1) providing a fibrous material dispersion;
2) mixing the fibrous material dispersion, a gelatin solution, and transglutaminase to obtain a precursor solution, and subjecting the precursor solution to pre-crosslinking and freeze-crosslinking in sequence, to obtain a crosslinked hydrogel;
3) mixing the crosslinked hydrogel with a first salt solution to perform salting-out and obtain a salting-out hydrogel;
4) stretching the salting-out hydrogel to obtain a stretched hydrogel; and
5) mixing the stretched hydrogel with a second salt solution and performing curing, to obtain the anisotropic hydrogel.

The physical crosslinking effect is present in the fibrous material dispersion.

Unless otherwise specified, the raw materials used in the present disclosure are commercially available products.

The present disclosure further provides a fibrous material dispersion.

In the present disclosure, the physical crosslinking effect present in the fibrous material dispersion includes, but is not limited to, hydrophobic association, hydrogen bonding, ionic bonding, and host-guest interaction.

In the present disclosure, the mass concentration of the fibrous material dispersion is preferably in a range of 1-10%, specifically 1%, 2%, 3%, 4%, 5%, 6%, 7%, 8%, 9%, or 10%. The fibrous material in the fibrous material dispersion preferably includes cellulose or protein isolate amyloid fibrils.

In some embodiments, when protein isolate amyloid fibrils are employed as the fibrous material in the fibrous material dispersion, a preparation method of the protein isolate amyloid fibril dispersion includes:
adding protein isolate into an ethanol-water system, adjusting the pH to an acidic value, and performing water-bath heating, to obtain the protein isolate amyloid fibril dispersion. In the present disclosure, the protein isolate is preferably pea protein isolate or soy protein isolate. In the present disclosure, the volume concentration of ethanol in the ethanol-water system is preferably in a range of 0-50%, specifically 0%, 10%, 20%, 30%, 40%, or 50%. The volume concentration of ethanol in the ethanol-water system can be used to control the morphology of the protein isolate amyloid fibrils, thereby regulating the microscopic morphology of the final hydrogel. In some embodiments, mixing the protein isolate with the ethanol-water system is performed under magnetic stirring. In the present disclosure, the pH is preferably in a range of 1.5-3, further preferably 2. The reagent used to adjust the pH to an acidic value is preferably hydrochloric acid solution, with a concentration of 3 mol/L. In the present disclosure, the water-bath heating temperature is preferably in a range of 70-90° C., specifically 70° C., 75° C., 80° C., 85° C., or 90° C. The heating time is preferably in a range of 20-40 hours, further preferably 30 hours, specifically 20 hours, 24 hours, 30 hours, 36 hours, or 40 hours.

In some embodiments, when the protein isolate is pea protein isolate, after mixing the protein isolate with the ethanol-water system and before adjusting the pH to an acidic value, the preparation method of the protein isolate amyloid fibril dispersion further includes removing precipitates. The precipitates are removed by centrifugation at a speed of 5000-7000 rpm, preferably 6000 rpm, and for 10-30 minutes, preferably 20 minutes.

In some embodiments, when the protein isolate is soy protein isolate, after adjusting the pH to an acidic value and before performing water-bath heating, the preparation method of the protein isolate amyloid fibril dispersion further includes performing hydration and removing precipitates. The hydration is performed in a refrigerator at 4° C. for 6-10 hours, preferably 8 hours. The precipitate is removed by centrifugation at a speed of 5000-7000 rpm, preferably 6000 rpm, and for 10-30 minutes, preferably 20 minutes.

In some embodiments, when the fibrous material is cellulose, the preparation method of the cellulose dispersion preferably includes: dispersing cellulose in water to obtain the cellulose dispersion. The water is preferably deionized water.

In some embodiments, after obtaining the fibrous material dispersion, the fibrous material dispersion, the gelatin solution, and transglutaminase, are mixed to obtain the precursor solution, and the precursor solution is subjected to pre-crosslinking and freeze-crosslinking in sequence, to obtain the crosslinked hydrogel.

In some embodiments, the mass concentration of the gelatin solution is preferably in a range of 5-20%, specifically 5%, 10%, 15%, or 20%. The preparation method of the gelatin solution preferably includes mixing gelatin with water and performing magnetic stirring to obtain the gelatin solution. The water is preferably deionized water.

In some embodiments, the transglutaminase is preferably used in the form of a transglutaminase solution, with a mass concentration of 10%. The preparation method of the transglutaminase solution includes mixing transglutaminase with water and performing magnetic stirring to obtain the transglutaminase solution. The water is preferably deionized water.

In some embodiments, mixing the fibrous material dispersion, gelatin solution, and transglutaminase includes: mixing the fibrous material dispersion and gelatin solution, adjusting the pH to 6-8, and then sequentially adding water and the transglutaminase solution. The pH value may be 6, 7, or 8. In some embodiments, in the precursor solution, the mass concentration of the fibrous material is in a range of 0.5-8%, preferably 1-5%, specifically 0.5%, 1%, 1.5%, 2%, 2.5%, 3%, 3.5%, 4%, 4.5%, 5%, 5.5%, 6%, 6.5%, 7%, 7.5%, or 8%. The mass concentration of gelatin is preferably in a range of 3-10%, specifically 3%, 4%, 5%, 6%, 7%, 8%, 9%, or 10%. The mass ratio of transglutaminase to gelatin ranges from 1:1 to 1:12, specifically 1:1, 1:2, 1:3, 1:4, 1:5, 1:6, 1:7, 1:8, 1:9, 1:10, 1:11, or 1:12.

In some embodiments, the pre-crosslinking is preferably performed at a temperature of 40-60° C., specifically 40° C., 45° C., 50° C., 55° C., or 60° C., and the pre-crosslinking is preferably performed for 2-30 minutes, specifically 2 minutes, 5 minutes, 10 minutes, 15 minutes, 20 minutes, 25 minutes, or 30 minutes. In some embodiments, the pre-crosslinking enables chemical crosslinking between a portion of the gelatin and the fibrous material. If all of the gelatin and the fibrous material are fully crosslinked, the molecular chains become immobilized and cannot be stretched later. Therefore, only partial crosslinking can ensure both the stretchability and stability of the material in subsequent processes without dissolution.

In some embodiments, the freeze-crosslinking is preferably performed at a temperature of 4° C., and the freeze-crosslinking is preferably performed for 1-3 hours, specifically 1 hour, 2 hours, or 3 hours. The freeze-crosslinking is preferably performed in a refrigerator. In the present disclosure, gelatin is a thermosensitive macromolecule; as the temperature decreases, the gelatin solution transitions from a liquid state to a solid state, and this transition is reversible, allowing the solution to return to a liquid state when the temperature increases. The freeze-crosslinking enables the solution to solidify and form a shape, facilitating subsequent salting-out operations.

In some embodiments, after obtaining the crosslinked hydrogel, the crosslinked hydrogel is mixed with the first salt solution, and salting-out is performed to obtain the salting-out hydrogel.

In some embodiments, the salt in the first salt solution includes one or more of sodium chloride, sodium sulfate, and sodium citrate, preferably sodium citrate. The mass concentration of the first salt solution is in a range of 15-40%, preferably 20-35%, specifically 15%, 20%, 25%, 30%, 35%, or 40%. In one embodiment, the first salt solution is a sodium citrate aqueous solution with a mass concentration of 30%.

In some embodiments, the volume ratio of the crosslinked hydrogel to the first salt solution ranges from 1:50 to 1:500.

In some embodiments, the salting-out is performed for 0.5-2 hours, preferably 1 hour, specifically 0.5 hour, 1 hour, 1.5 hours, or 2 hours. The salting-out is preferably performed at room temperature, requiring neither additional heating nor cooling.

In some embodiments, after obtaining the salting-out hydrogel, the salting-out hydrogel is stretched to obtain a stretched hydrogel.

In some embodiments, the salting-out hydrogel is preferably stretched to 1-6 times its original length, specifically to 1×, 2×, 3×, 4×, 5×, or 6× the original length. The stretching is preferably performed for 5 -15 minutes, specifically 5 minutes, 10 minutes, or 15 minutes.

In some embodiments, after obtaining the stretched hydrogel, the stretched hydrogel is mixed with the second salt solution, and curing is performed to obtain the anisotropic hydrogel.

In some embodiments, the salt in the second salt solution preferably includes sodium citrate. The mass concentration of the second salt solution is in a range of 15-40%, preferably 20-35%, specifically 15%, 20%, 25%, 30%, 35%, or 40%.

In some embodiments, the curing is preferably performed for 10-20 hours, specifically 10 hours, 12 hours, 15 hours, 18 hours, or 20 hours, and the curing is preferably performed at room temperature, requiring neither additional heating nor cooling.

The present disclosure further provides the anisotropic hydrogel prepared by the preparation method described above. The surface of the anisotropic hydrogel exhibits directional groove-like structures.

The present disclosure further provides the use of the anisotropic hydrogel described above in the food field.

In some embodiments, the food field is preferably cultured meat preparation.

In the present disclosure, the surface of the anisotropic hydrogel exhibits directional groove-like structures, enabling cells to grow in an oriented manner on the surface, thereby forming fibrous cultured meat. The resulting cultured meat has a texture close to that of real meat.

The following examples are provided to describe in detail the anisotropic hydrogel, its preparation method, and its use according to the present disclosure. However, these examples should not be understood as limiting the scope of the present disclosure.

### Example 1: Preparation of Pea/Soy Protein Isolate Amyloid Fibril Dispersion

### Preparation of Pea Protein Isolate Amyloid Fibril (PPIF) Dispersion

Different amounts of pea protein isolate were weighed and added to 50 mL of deionized water or ethanol-water solutions with different volume concentrations (10%, 20%, 30%, 40%, 50%). The mixtures were fully dissolved under magnetic stirring and then centrifuged at 6000 rpm for 20 minutes to remove precipitates. The pH was adjusted to 2 using 3 mol/L hydrochloric acid, and water bath heating was performed at 80° C. for 30 hours. As a result, pea protein isolate amyloid fibril dispersions with a mass concentration of 10% were obtained.

### Preparation of Soy Protein Isolate Amyloid Fibril (SPIF) Dispersion

Different amounts of soy protein isolate were weighed and added to 50 mL of deionized water or ethanol-water solutions with different volume concentrations (10%, 20%, 30%, 40%, 50%). The mixtures were fully dissolved under magnetic stirring. The pH was adjusted to 2 using 3 mol/L hydrochloric acid, followed by hydration in a refrigerator at 4° C. for 8 hours. Centrifugation was performed at 6000 rpm for 20 minutes to remove precipitates, followed by water bath heating at 80° C. for 30 hours. Soy protein isolate amyloid fibril dispersions with a mass concentration of 10% were thus obtained.

The morphology of the protein isolate amyloid fibrils in the dispersions prepared under different conditions obtained in Example 1 was observed using scanning transmission electron microscopy (JEM-1400flash). The results are shown in FIG. 1. In FIG. 1, panel A, from left to right, shows PPIF obtained using ethanol-water solutions with ethanol volume concentrations of 0%, 10%, 20%, 30%, 40%, and 50%; panel B, from left to right, shows SPIF obtained using ethanol- water solutions with ethanol volume concentrations of 0%, 10%, 20%, 30%, 40%, and 50%. As shown in FIG. 1, the morphology of PPIF and SPIF gradually changed from rigid to flexible and then to a network structure as the ethanol volume concentration increased. Straight fibers were considered rigid fibers, whereas curved and soft fibers were considered flexible fibers. Specifically, the fibers of PPIF-0% and SPIF-0% were relatively straight. With the increase of ethanol volume concentration, the fibers gradually become curved, and further increase in ethanol concentration led to deeper fiber entanglement, forming a network structure. Specifically, the rigidity or flexibility of the fibers was determined by comparing the contour length and persistence length of the fibers. The contour length refers to the end-to-end length of the polymer along its physical contour. The persistence length is an intrinsic property of the polymer and can be estimated by calculating the mean-squared end-to-end distance between contour segments, which is one of the most practical and widely used methods. When the contour length is much greater than the persistence length, the fiber is considered a flexible chain; when the contour length is less than the persistence length, the fiber is considered a rigid chain.

### Example 2: Construction of Stretchable Pea/Soy Protein Isolate Amyloid Fibril-Gelatin Hydrogels Via Salting-Out

### Preparation of Pea Protein Isolate Amyloid Fibril (PPIF) Dispersion

Different amounts of pea protein isolate were weighed and added to 50 mL of water (i.e., ethanol-water solution with ethanol volume concentration of 0%). The mixtures were fully dissolved under magnetic stirring and centrifuged at 6000 rpm for 20 minutes to remove precipitates. The pH was adjusted to 2 using 3 mol/L hydrochloric acid, followed by water bath heating at 80° C. for 30 hours. Pea protein isolate amyloid fibril dispersions with a mass concentration of 10% were thus obtained.

### Preparation of Soy Protein Isolate Amyloid Fibril (SPIF) Dispersion

Different amounts of soy protein isolate were weighed and added to 50 mL of water (i.e., ethanol-water solution with ethanol volume concentration of 0%). The mixtures were fully dissolved under magnetic stirring, and the pH was adjusted to 2 using 3 mol/L hydrochloric acid. Hydration was performed in a refrigerator at 4° C. for 8 hours, followed by centrifugation at 6000 rpm for 20 minutes to remove precipitates, and then water bath heating at 80° C. for 30 hours. Soy protein isolate amyloid fibril dispersions with a mass concentration of 10% were obtained.

### Preparation of Gelatin Solution

Gelatin was weighed and added to 50 mL of deionized water. The mixture was fully dissolved under magnetic stirring, yielding a 20% mass concentration gelatin solution.

### Preparation of Stretchable Pea/Soy Protein Isolate Amyloid Fibril-Gelatin Hydrogel

The PPIF dispersion or SPIF dispersion was mixed with gelatin solution under magnetic stirring at room temperature for 1 hour. The pH was adjusted to 7 using 3 mol/L hydrochloric acid, and deionized water was added to obtain a precursor solution. In the precursor solution, the mass concentration of pea/soy protein isolate amyloid fibrils was 1%, and the mass concentration of gelatin was 2%. The precursor solution was placed in a refrigerator at 4° C. for 1 hour to perform freeze-crosslinking, yielding a crosslinked hydrogel. The crosslinked hydrogel was immersed in a 30% mass concentration sodium citrate solution, wherein a volume ratio of the crosslinked hydrogel to sodium citrate solution was 1:100. The crosslinked hydrogel was incubated at room temperature for 1 hour, yielding a salting-out hydrogel. The salting-out hydrogel was stretched to 5 times its original length and the stretching was maintained for 10 minutes, yielding a stretched hydrogel. The stretched hydrogel was then immersed in a 30% mass concentration sodium citrate solution, wherein a volume ratio of the stretched hydrogel to the sodium citrate solution was 1:100, and cured for 12 hours, resulting in anisotropic gelatin-PPIF and anisotropic gelatin-SPIF hydrogels.

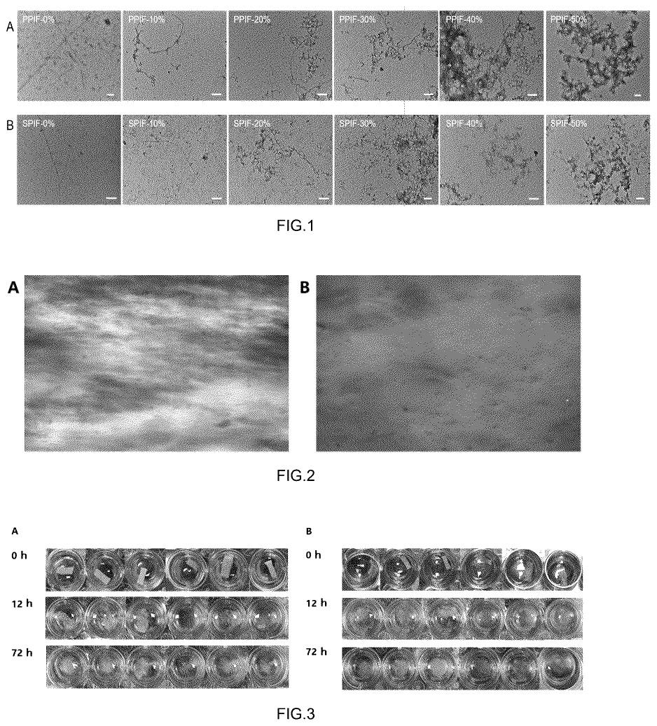

The polarization behavior of the anisotropic gelatin-PPIF and anisotropic gelatin-SPIF hydrogels after stretching was observed using a polarizing microscope. The results are shown in FIG. 2. In FIG. 2, panel A shows the polarizing microscopy images of the anisotropic gelatin-PPIF hydrogel, and panel B shows the polarizing microscopy images of the anisotropic gelatin-SPIF hydrogel. As shown in FIG. 2, the stretched hydrogels exhibited polarization phenomena. Anisotropic materials typically show birefringence, where light exhibits different refractive indices in different directions within the material. When a beam of unpolarized light passes through an anisotropic material, it is split into two polarized beams that propagate along the fast axis and slow axis, producing bright and dark contrast patterns under polarizing microscopy.

The obtained anisotropic gelatin-PPIF and anisotropic gelatin-SPIF hydrogels were immersed in culture medium and incubated in incubators at 27° C. and 37° C. The hydrogel state at different incubation time points was recorded. The results are shown in FIG. 3, which presents optical photographs of the anisotropic gelatin-PPIF and anisotropic gelatin-SPIF hydrogels at 27° C. and 37° C. over different incubation durations. In FIG. 3, panel A shows the optical photographs of the anisotropic gelatin-PPIF hydrogel at 27° C. and 37° C. over different incubation durations, and panel B shows the optical photographs of the anisotropic gelatin-SPIF hydrogel at 27° C. and 37° C. over different incubation durations. As shown in FIG. 3, under these conditions, the anisotropic gelatin-protein isolate amyloid fibril hydrogels dissolved in the culture medium over time.

### Example 3: Synergistic Construction of Anisotropic Pea/Soy Protein Isolate Amyloid Fibril-Gelatin Hydrogels Via Enzymatic Crosslinking and Salting-Out

### Preparation of Pea Protein Isolate Amyloid Fibril (PPIF) Dispersion

Different amounts of pea protein isolate were weighed and added to an ethanol-water solution with an ethanol volume concentration of 30%. The mixtures were fully dissolved under magnetic stirring and centrifuged at 6000 rpm for 20 minutes to remove precipitates. The pH was adjusted to 2 using 3 mol/L hydrochloric acid, followed by water bath heating at 80° C. for 30 hours, yielding pea protein isolate amyloid fibril dispersions with a mass concentration of 10%.

### Preparation of Soy Protein Isolate Amyloid Fibril (SPIF) Dispersion

Different amounts of soy protein isolate were weighed and added to 30 mL of ethanol-water solution with an ethanol volume concentration of 30%. The mixtures were fully dissolved under magnetic stirring, and the pH was adjusted to 2 using 3 mol/L hydrochloric acid. Hydration was performed in a refrigerator at 4° C. for 8 hours, followed by centrifugation at 6000 rpm for 20 minutes to remove precipitates, and then water bath heating at 80° C. for 30 hours, yielding soy protein isolate amyloid fibril dispersions with a mass concentration of 10%.

### Preparation of Gelatin Solution

Gelatin was weighed and added to 50 mL of deionized water. The mixture was fully dissolved under magnetic stirring, yielding a 20% mass concentration gelatin solution.

### Preparation of Transglutaminase (TG) Solution

1 g of transglutaminase was weighed and added to 10 mL of deionized water. The mixture was fully dissolved under magnetic stirring, yielding a 10% mass concentration transglutaminase solution.

### Preparation of Stretchable Pea/soy Protein Isolate Amyloid Fibril-gelatin Hydrogel

The PPIF or SPIF dispersion was mixed with gelatin solution under magnetic stirring at room temperature for 1 hour. The pH was adjusted to 7 using 3 mol/L hydrochloric acid, deionized water was added, and then the transglutaminase solution was incorporated to obtain a precursor solution. In the precursor solution, the mass concentration of PPIF or SPIF was 1%, the mass concentration of gelatin was 5%, and the mass ratio of transglutaminase to gelatin was 1:10. The precursor solution was subjected to pre-crosslinking in a 50° C. water bath for 5 minutes to obtain a pre-crosslinked hydrogel, which was then cooled in a refrigerator at 4° C. for 1 hour to obtain a crosslinked hydrogel. The crosslinked hydrogel was immersed in a 30% mass concentration sodium citrate solution at room temperature for 1 hour to obtain a salting-out hydrogel.

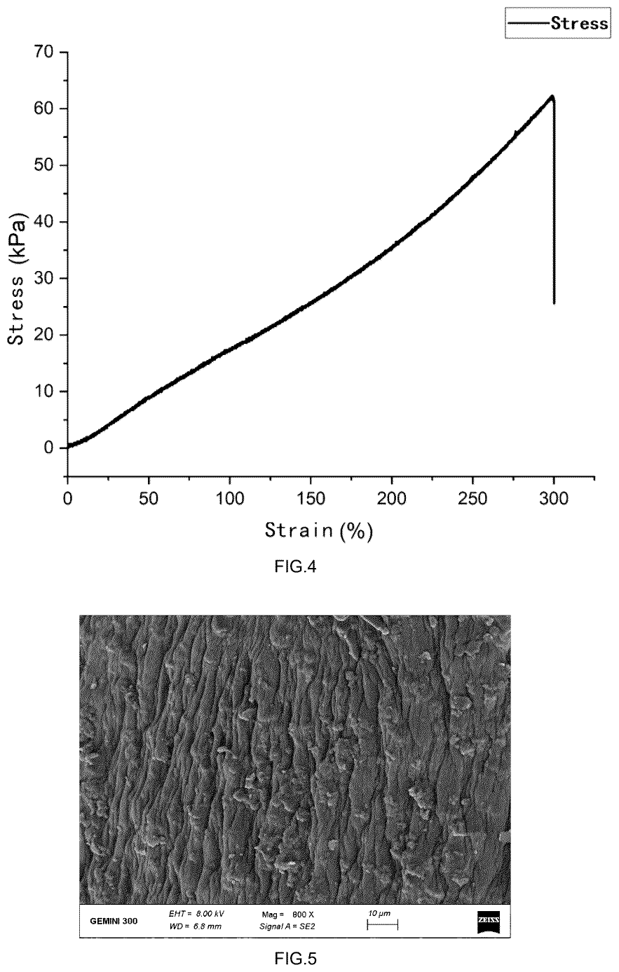

The salting-out hydrogel prepared from SPIF with an ethanol concentration of 30% was stretched to 3 times its original length using a texture analyzer, and the stress-strain curve was recorded in FIG. 4. As shown in FIG. 4, when the salting-out hydrogel is stretched to 3 times its original length, the stress of the salting-out hydrogel reached 62.29 kPa.

### Example 4: Synergistic Construction of Anisotropic Pea/Soy Protein Isolate Amyloid Fibril-Gelatin Hydrogels Via Enzymatic Crosslinking and Salting-Out

### Preparation of Pea Protein Isolate Amyloid Fibril (PPIF) Dispersion

Different amounts of pea protein isolate were weighed and added to an ethanol-water solution with an ethanol volume concentration of 10%. The mixtures were fully dissolved under magnetic stirring and centrifuged at 6000 rpm for 20 minutes to remove precipitates. The pH was adjusted to 2 using 3 mol/L hydrochloric acid, followed by water bath heating at 80° C. for 30 hours, yielding pea protein isolate amyloid fibril solutions with a mass concentration of 10%.

### Preparation of Soy Protein Isolate Amyloid Fibril (SPIF) Dispersion

Different amounts of soy protein isolate were weighed and added to 50 mL of ethanol-water solution with an ethanol volume concentration of 10%. The mixtures were fully dissolved under magnetic stirring, and the pH was adjusted to 2 using 3 mol/L hydrochloric acid. Hydration was performed in a refrigerator at 4° C. for 8 hours, followed by centrifugation at 6000 rpm for 20 minutes to remove precipitates, and then water bath heating at 80° C. for 30 hours, yielding soy protein isolate amyloid fibril solutions with a mass concentration of 10%.

### Preparation of Gelatin Solution

Gelatin was weighed and added to 50 mL of deionized water. The mixture was fully dissolved under magnetic stirring, yielding a 20% mass concentration gelatin solution.

### Preparation of Transglutaminase (tg) Solution

1 g of transglutaminase was weighed and added to 10 mL of deionized water. The mixture was fully dissolved under magnetic stirring, yielding a 10% mass concentration transglutaminase solution.

### Preparation of Stretchable Pea/soy Protein Isolate Amyloid Fibril-gelatin Hydrogel

The PPIF or SPIF solution was mixed with gelatin solution under magnetic stirring at room temperature for 1 hour. The pH was adjusted to 7 using 3 mol/L hydrochloric acid, deionized water was added, and then the transglutaminase solution was incorporated to obtain a precursor solution. In the precursor solution, the mass concentration of PPIF or SPIF was 1%, the mass concentration of gelatin was 5%, and the mass ratio of transglutaminase to gelatin was 1:10. The precursor solution was subjected to pre-crosslinking in a 50° C. water bath for 5 minutes to obtain a pre-crosslinked hydrogel, which was then cooled in a refrigerator at 4° C. for 1 hour to obtain a crosslinked hydrogel. The crosslinked hydrogel was immersed in a 30% mass concentration sodium citrate solution at room temperature for 1 hour to obtain a salting-out hydrogel. The salting-out hydrogel was stretched to 5 times its original length, and the stretching was maintained for 10 minutes, yielding a stretched hydrogel. The stretched hydrogel was then immersed in a 30% mass concentration sodium citrate solution and cured for 12 hours to obtain anisotropic gelatin-PPIF and anisotropic gelatin-SPIF hydrogels.

The surface morphology of the anisotropic gelatin-PPIF hydrogel was observed using scanning electron microscopy. The results are shown in FIG. 5. As shown in FIG. 5, the surface of the stretched hydrogel exhibited directional groove-like structures, which is a typical morphology of anisotropic hydrogels.

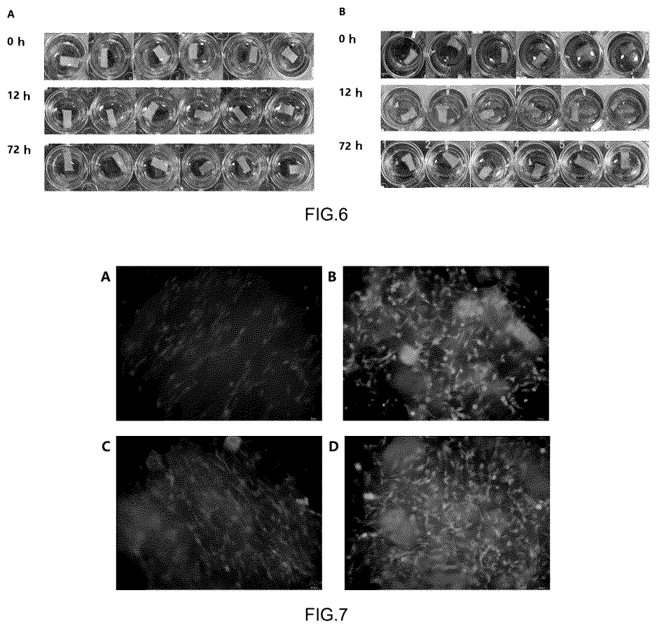

The anisotropic gelatin-PPIF and gelatin-SPIF hydrogels were immersed in culture medium and incubated in incubators at 27° C. and 37° C. The hydrogel state at different incubation time points was recorded in FIG. 6, which presents optical photographs of the anisotropic gelatin-protein isolate amyloid fibril hydrogels at 27° C. and 37° C. over different incubation durations. In FIG. 6, panel A shows the optical photographs of the anisotropic gelatin-PPIF hydrogel at 27° C. and 37° C. over different incubation durations, and panel B shows the optical photographs of the anisotropic gelatin-SPIF hydrogel at 27° C. and 37° C. over different incubation durations. As shown in FIG. 6, under these conditions, the enzymatically crosslinked hydrogels remained stable when immersed in the culture medium, showing no significant changes.

The stretched anisotropic gelatin-PPIF and anisotropic gelatin-SPIF hydrogels, as well as the untreated salting-out hydrogels, were used for cell culture. Fluorescence microscopy was used to observe cell morphology, and the results are shown in FIG. 7. In FIG. 7, panel A shows cell morphology of the cells cultured on the stretched anisotropic gelatin-PPIF hydrogel, panel B shows cell morphology of the cells cultured on the unstretched salting-out PPIF-gelatin hydrogel, panel C shows cell morphology of the cells cultured on the stretched anisotropic gelatin- SPIF hydrogel, and panel D shows cell morphology of the cells cultured on the unstretched salting-out SPIF-gelatin hydrogel. As shown in FIG. 7, cells grew in a random and disordered manner on the untreated hydrogels (see panels B and D), while cells exhibited directional alignment on the stretched anisotropic hydrogels (see panels A and C).

The above-described embodiments are merely preferred examples of the present disclosure. It should be noted that those skilled in the art can make various modifications and refinements without departing from the principles of the present disclosure. Such modifications and refinements shall also fall within the scope of the present disclosure.

What is claimed is:

1. A method for preparing an anisotropic hydrogel, comprising:
1) providing a fibrous material dispersion;
2) mixing the fibrous material dispersion, a gelatin solution, and transglutaminase to obtain a precursor solution, and subjecting the precursor solution to pre-crosslinking and freeze-crosslinking in sequence, to obtain a crosslinked hydrogel;
3) mixing the crosslinked hydrogel with a first salt solution to perform salting-out and obtain a salting-out hydrogel;
4) stretching the salting-out hydrogel to obtain a stretched hydrogel; and
5) mixing the stretched hydrogel with a second salt solution and performing curing, to obtain the anisotropic hydrogel;
wherein a physical crosslinking effect is present in the fibrous material dispersion;
wherein the pre-crosslinking is carried out at 40-60° C. for 2-30 minutes;
the freeze-crosslinking is carried out at 4° C. for 1-3 hours; and
a fibrous material in the fibrous material dispersion is protein isolate amyloid fibrils, and the protein isolate amyloid fibrils are pea protein isolate amyloid fibrils or soy protein isolate amyloid fibrils.

2. The method of claim 1, wherein when the protein isolate amyloid fibrils are employed as the fibrous material in the fibrous material dispersion, a preparation method of a protein isolate amyloid fibril dispersion comprises:
adding a protein isolate into an ethanol-water system, adjusting the pH to acidic, and performing water bath heating to obtain the protein isolate amyloid fibril dispersion; wherein
the protein isolate comprises pea protein isolate or soy protein isolate;
a volume concentration of ethanol in the ethanol-water system is 0-50%;
the acidic pH is in a range of 1.5-3; and
the water bath heating is carried out at 70-90° C. for 20-40 hours.

3. The method of claim 2, wherein when the protein isolate is soy protein isolate, after adjusting the pH to acidic and before performing water bath heating, the preparation method of the protein isolate amyloid fibril dispersion further comprises: performing hydration and removing precipitates;
wherein the hydration is carried out at 2-8° C. for 6-10 hours.

4. The method of claim 1, wherein in the precursor solution, a mass concentration of the fibrous material is in a range of 0.5-8%, a mass concentration of gelatin is in a range of 3-10%, and a mass ratio of transglutaminase to gelatin ranges from 1:1 to 1:12.

5. The method of claim 1, wherein a salt in the first salt solution comprises one or more of sodium chloride, sodium sulfate, and sodium citrate;
a mass concentration of the first salt solution is in a range of 15-40%; and
the salting-out is carried out for 0.5-2 hours.

6. The method of claim 1, wherein the salting-out hydrogel is stretched to 1-6 times its original length.

7. The method of claim 1, wherein a salt in the second salt solution comprises sodium citrate;
a mass concentration of the second salt solution is in a range of 15-40%; and
the curing is carried out for 10-20 hours.

8. An anisotropic hydrogel, prepared by the method of claim 1.

9. A method for preparing a food, comprising: incorporating the anisotropic hydrogel of claim 8.

10. The method of claim 9, wherein the food is cultured meat.
